(19) United States
(12) Patent Application Publication
(10) Pub. No.: US 2006/0047188 A1
(43) Pub. Date: Mar. 2, 2006

(54) Method and system for triage of emergency patients

(76) Inventor: J. Stephen Bohan, Vineyard Haven, MA (US)

Correspondence Address: BARLOW, JOSEPHS & HOLMES, LTD., 101 DYER STREET, 5TH FLOOR, PROVIDENCE, RI, 02903, US

(21) Appl. No.: 11/196,001

(22) Filed: Aug. 3, 2005

Related U.S. Application Data
(60) Provisional application No. 60/605,009, filed on Aug. 27, 2004.

(51) Int. Cl. A61B 5/00; G06F 19/00

(52) U.S. Cl. 600/300; 600/301; 705/3

(57) ABSTRACT

A method and system for automated patent intake and triage in the context of an emergency care center is provided. The system consists of a kiosk computer terminal, which is placed in a receiving area within the emergency treatment center and preferably includes a touch screen interface, a variety of physiological monitoring devices and software that collects and assesses the user input and physiological data to calculate the severity of the patient's condition. This allows automated triage and prioritization of patients thereby freeing health care professional resources to perform other patient care functions.

### CROSS-REFERENCE TO RELATED APPLICATIONS

This application is related to and claims priority from earlier filed U.S. Provisional Patent Application No. 60/605,009 filed Aug. 27, 2004, the contents of which are incorporated herein by reference.

### BACKGROUND OF THE INVENTION

The present invention relates generally to a device and system for automated patient intake and monitoring. More specifically the present invention is directed to an automated system for assessing patients upon their arrival to an emergency treatment center, thereby streamlining and reducing the amount of resources required to perform the patient intake and prioritization process.

One of the greatest difficulties being faced today in the medical community is the rapidly rising cost associated with providing health care. Where these costs become particularly problematic is in the context of emergency treatment centers wherein the number of patients being treated is dramatically increasing with each passing year. More and more, people are turning to emergency care centers rather than a primary care physician or specialist to treat a variety of illnesses thereby further increasing the burden borne by the emergency treatment centers across the country causing the emergency care centers to become clogged with literally thousands of patients that have non-urgent medical conditions. This is problematic because typically the cost for treating a patient in the emergency treatment center is much greater than the cost associated with doctor's office consultations because of the large amount of staff and equipment required to maintain a full service emergency treatment center.

At the same time, the administrative demands related to medical record keeping, billing and managing any medical practice have also become more burdensome. In particular, health care providers must be thorough and keep detailed records of any medical exams, treatments and patient histories to accurately document observations and services that have been provided. In order to continue to provide ha high quality level of service and care while controlling the escalating costs, it is imperative that health care providers continually find innovative ways to provide health services efficiently and cost effectively.

In this regard, a number of computerized tracking systems and software tools are available to assist physicians in conducting medical diagnoses and medical record keeping. However, while these tools provide assistance in storing and managing the data after it has been collected, most of these systems still require that the process begin with a medical professional consultation, wherein the medical professional makes an initial assessment of the patient to arrive at an initial tentative diagnosis and then proceeds to collect medical observations which support, or clarify the tentative diagnosis. Another limitation of many software systems used for data collection and management is that they while they provide guidance in the assessment process and storage facilities for collected data, they do not serve to assist in managing the prioritization of patients and the overall workflow within the hospital.

These limitations become particularly acute in the context of an emergency treatment center where 115 million patients are treated in the United States on an annual basis. Upon arrival at the emergency care center, each and every case must be sorted by the severity of the patient's condition in a procedure referred to as triage. Normally, when a patient arrives at an emergency treatment center, the procedure begins with a manually performed intake consultation with a nurse. Typically, the nurse asks the patient a few questions in order to immediately assess the patient and determine the relative severity of the patient's condition. In addition to asking questions, the medical professional typically takes the patient's vital signs. Based on this initial evaluation of the patient's symptoms and vital signs, the medical professional then makes a decision about the criticality of the patient's condition and assigns a criticality or triage rating to each patient. Patients in the emergency treatment center are then placed into a prioritized order for receiving medical attention based on the medical professional's assigned critically of the patient's condition It can be appreciated that those patient's with the most critical conditions are seen immediately while those with the least severe conditions are placed lower in the list where they must wait for treatment. This manual triage process is time consuming and expensive in that it consumes medical professional resources that can be better used in the actual delivery of care.

Further, the process of emergency treatment center triage is particularly difficult, even for well-trained and experienced medical professionals because of the limited amount of information obtained and the short window of time during which the initial consultation is conducted. Based on this truncated examination of the patient, the medical professional must quickly assess and assign priority to the patient's case. Additional complexity is added to the triage process as the emergency treatment center becomes busier and the medical professional is required to increase the rate of patient triage while operating based on the medical professional's retained knowledge. In other words, the triage process is typically conducted based on the medical professional's memory, without clear guidelines and without a well established and readily available decision tree that can be employed to make sure they ask the right questions every time and then properly evaluate the patient's condition based on the answers to their questions and based on their observations of the patient's physical condition. Even in cases where the medical professional has sufficient time available to perform a comprehensive assessment, including obtaining the chief complaint, past medical and surgical history, medications and allergies, and spends a great deal of time acquiring this information, the medical professional may still wonder whether they made the right triage decision about a patient.

Once a triage decision is made, the patient is sent back to an emergency waiting area where they remain seated while awaiting their turn to be seen by a doctor for treatment. During this waiting period, in the present treatment cycle, there is no provision for follow up consultations or patient monitoring in order to determine whether the patient's condition has changed or deteriorated. Should the initial triage assessment be incorrect, the present system is lacking in a feed back loop to assist in identifying an incorrect assessment that may be cause to readjust the priority assigned during the initial triage assessment.

The consequence is that errors in triage decisions can result in a longer waiting period for a patient who has a serious or life threatening condition that is badly in need of immediate medical care, while other less critical patients are given medical attention. Obviously, such an error in triage of patients is undesirable both from the patient's standpoint who may need immediate care and who cannot get it because emergency department personnel did not properly identify the critical nature of his condition and from the hospital's and physician's standpoints because both are morally dedicated to helping people and are both financially at risk for failing to provide the proper level of care to patients.

There is therefore a need for a method and system whereby the initial intake, assessment and triage of a patient arriving at an emergency treatment center is automated in a manner that increases the rate patient of intake while also enhancing the overall accuracy of patient assessment and triage prioritization. Further, there is a need of a method and system that automates patient intake thereby assisting in workflow management and freeing valuable medical professional resources for use in providing direct patient care by making use of the accuracy, reliability and reproducibility that is inherent in computer technology.

### BRIEF SUMMARY OF THE INVENTION

In this regard, the present invention provides a novel method and system for automated patent intake and triage. The system consists of a computer terminal, which is placed in a receiving area within the emergency treatment center. The computer terminal is preferably configured to include a touch screen interface, a variety of physiological monitoring devices and software that collects and assesses the user input and physiological data to calculate the severity of the patient's condition.

In the preferred embodiment, the present invention utilizes an automated teller machine (ATM) style interface and a networked communication connection to collect user input in the form of patient biographical data, demographics, physiological data and the principal reason for their present visit to the emergency care center while also including access to the patient's medical history thereby providing the software component with background information that may be pertinent to correct diagnosis of the patient. Based on the collected data, the software ranks the severity of the patient's condition thereby providing a triage rating for each patient in the emergency treatment center. In this manner, the present invention addresses many of the above noted problems by providing a computer based triage system that helps eliminate most of the burden of triage decision making from the medical professional, freeing them for direct care delivery

In operation, the method of the present invention provides that upon entering the emergency care center, a patient is greeted by a health care professional that is certified in triage operations. The health care professional makes an initial and very brief determination as to whether the arriving patient requires immediate treatment. If the arriving patient does not warrant immediate treatment, the health care professional directs the patient to the automated system of the present invention, wherein the patient begins the automated triage process. The computer terminal guides the user through a series of questions to determine their background, language preference, medical history and chief complaint. The computer then also checks the patient's physiological data such as pulse, blood pressure, temperature and respiratory rate. This information is all analyzed by the proprietary software in the computer to assign an emergency severity index (ESI) level. All of the collected data including the ESI score is subsequently sent to a medical professional that reviews the data and approves it, at which time the network compiles the information and prints the patient chart and ranks the patients based on their ESI. The medical professional then manages patient flow according to ESI. In this manner a large portion of the initial triage process is automated while still allowing the medical professionals to apply their observational skills and experience in order to make the final determination regarding the patient's condition.

It is therefore an object of the present invention to provide a method and system whereby the initial intake, assessment and triage of a patient arriving at an emergency treatment center is automated in a manner that increases the rate patient of intake while also enhancing the overall accuracy of patient assessment and triage prioritization. It is a further object of the present invention to provide a method and system that automates patient intake thereby assisting in workflow management and freeing valuable medical professional resources for use in providing direct patient care. It is yet a further object of the present invention to provide a method and system whereby patient workflow management is automated in a manner that facilitates efficient allocation of staffing based on known levels of patient need.

These together with other objects of the invention, along with various features of novelty, which characterize the invention, are pointed out with particularity in the claims annexed hereto and forming a part of this disclosure. For a better understanding of the invention, its operating advantages and the specific objects attained by its uses, reference should be had to the accompanying drawings and descriptive matter in which there is illustrated a preferred embodiment of the invention.

### BRIEF DESCRIPTION OF THE DRAWINGS

In the drawings which illustrate the best mode presently contemplated for carrying out the present invention:

FIG. 1 is a schematic illustration of the system of the present invention; and

FIG. 2 is a flow chart depicting a preferred embodiment of the method of the present invention.

### DETAILED DESCRIPTION OF THE INVENTION

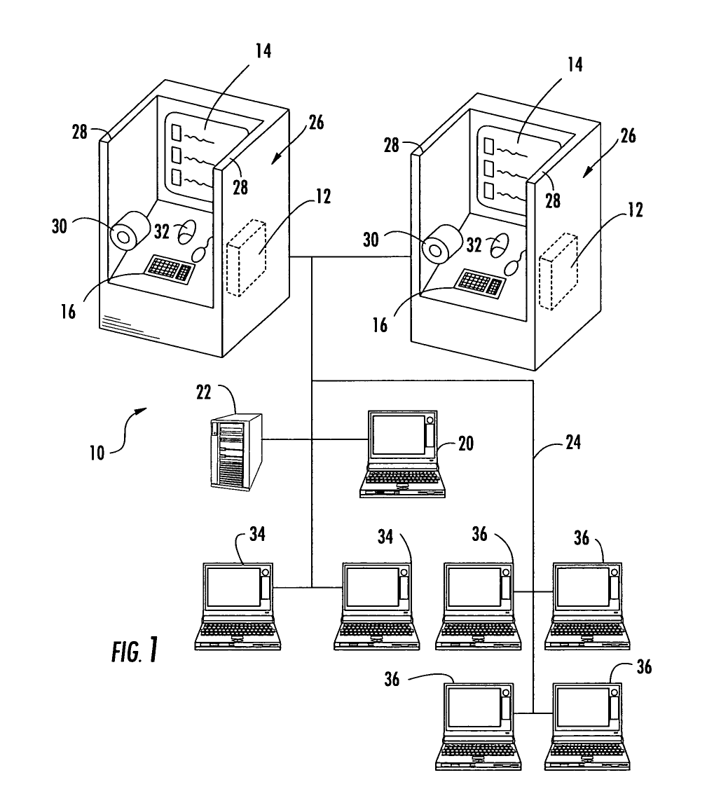

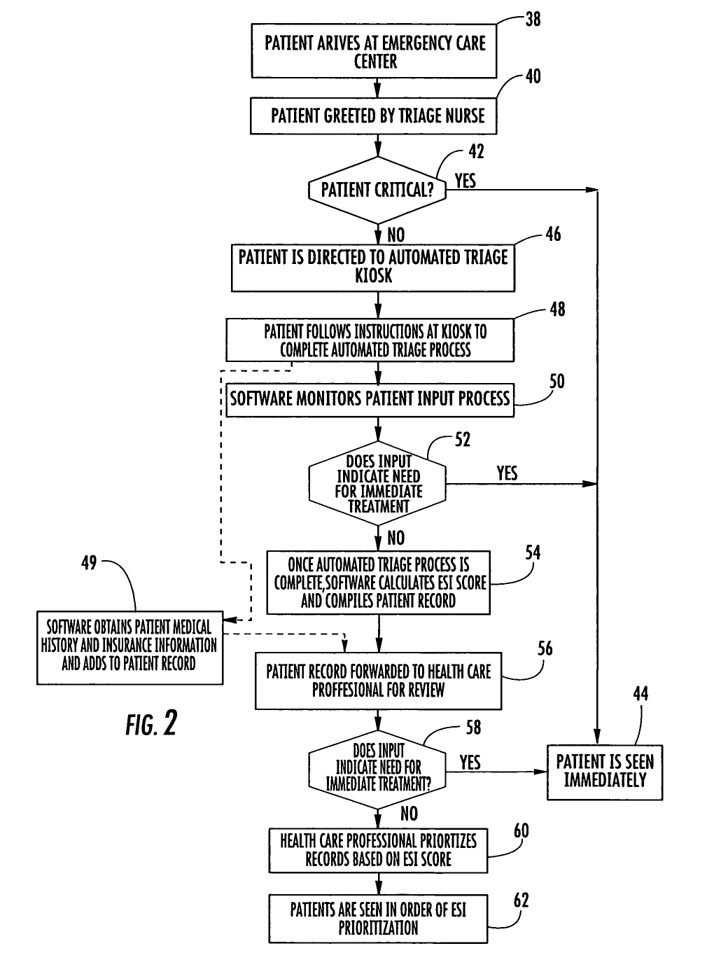

Now referring to the drawings, a schematic illustration of the system of the present invention is shown and generally illustrated at FIG. 1. Further, FIG. 2 shows a flow chart depicting the preferred embodiment of the method of the present invention.

Turning now to FIG. 1, as was stated above, the present invention provides a novel method and system for automated patient intake and triage. The system 10 generally consists of a computer processor device 12 that includes at least a monitor 14 and a user interface 16. The system 10 also preferably provides for the computer processor 12 to be connected to a local electronic communication network 18 wherein the computer processor 12 is capable of communicating with at least one other processor device 20 and at least one data storage device 22, both of which are also connected to the local network 18. Further, in one embodiment of the present invention, it is preferable that the local communication network 18 be accessible from remote locations via a wide area communications network 24 such as the Internet.

The computer processor device 12 may simply be an access terminal connected to the local area network 18 or may include a computer processor and limited local storage capacity. The computer processor device 12 for example may be in the form of an ATM type device or in the form of a personal computer. The basic hardware requirements associated with such a computer processor device 12 are well known and documented in the art such that an explanation of such hardware specific components need not be provided herein. In either case, the computer processor device 12 is installed in a kiosk type arrangement 26 in a manner that limits access to the computer processor device 12 except for the purpose of a user interacting with the user interface 16. Additionally, in order to comply with the requirements of the health information protection act (HIPA) the kiosk 26 may also be configured to include privacy screens 28 to block visibility of the user interface 16 and monitor 14 thereby preventing anyone but the intended user from viewing the monitor screen 14.

The user interface 16 in the system 10 of the preferred embodiment is in the form of a touch screen monitor 14 thereby providing the user of the system with visual on screen prompts and questions in a manner that allows the user to proceed through the triage process in an automated fashion. Similarly, rather than a touch screen monitor 14, the user interface may be in the form of a keyboard and/or mouse that are connected to the computer processor device 12 and configured for input and a monitor screen 14 to enable the user to view the prompts and questions.

Additional user input devices include various biometrics and/or physiological monitoring devices such as for example a sphygmomanometer (blood pressure cuff) 30, an infrared sensor array 32 for detecting pulse rate, blood oxygen levels and the patient's body temperature and a laser sensor to measure the patients respiratory rate. Further wile specific examples of instruments are provided above, the present invention anticipates using any suitable instrument for detecting physiological data including a sphygmomanometer, an infrared sensor array, a means for detecting pulse rate, a means for detecting blood oxygen levels, a means for detecting body temperature, a means to measure respiratory rate and combinations thereof such as are already well known in the medical arts. Each one of these devices are also connected to the computer processor device 12 and serve to supply their various readings and measurements to the computer processor device 12 for inclusion in the patient's file in conjunction with the other user supplied input.

It is anticipated that in the context of the system 10 of the present invention that there will be an array of any number of kiosks 26 each containing a discrete computer processor device 12 and a user interface 16 thereby allowing multiple patients to each input their triage information simultaneously as they arrive at the emergency care center.

Preferably, each of the computer processor devices 12 in the array of kiosks 26 is connected to the local area communication network 18. Through this connection, the computer processor devices 12 are enabled to communicate with a remote terminal 20, which is monitored by a medical professional. This communication link via the local area network 18 allows the discrete user input from each of the computer processor devices 12, including the user supplied information and the physiological data, to be transferred to the remote terminal 20 once the users have completed the required input in the triage process thereby allowing a medical professional to review the triage information input by the patients. Based on a review of the information provided, the medical professional assigns an emergency severity index (ESI) to each of the patient files, as will be further described in the process below, for the purpose of ranking the various patients that are arriving in the emergency care center based on the severity of their chief complaint in combination with their physiological information.

In addition to providing the opportunity for a medical professional to review and prioritize each of the patient cases upon entry to the emergency care center from a remote location, the local area network connection 18 may also allow the computer processor device 12 to communicate with a central database 22 for the purpose of accessing a patient's prior medical history that may be stored therein. Further, such a local area network communication link 18, in addition to providing for the linking of a patient's medical history with the triage information, allows the system to link various medical care provider and insurance information to the triage record providing a complete picture and history of the patient in a manner that streamlines the record keeping and patient intake process from an administrative perspective.

Finally, the communication link from the computer processor device 12 in conjunction with both the local area network 18 and a connection to a wide area network 24 provides a broad ability to manage emergency care center workflow and staffing requirements. By providing a connection between the emergency care center local area network 18 and additional computers 34 located throughout a hospital environment or an array of computers 36 connected via a wide area network 24 such as the Internet, a large number of authorized health care professionals can monitor the status of the emergency care center. In this manner, when the emergency care center is handling a low volume of patients, health care professionals can attend to duties elsewhere. As patient volume increases in the emergency care and the need for staffing increases, health care professionals who are monitoring patient volume via both the local area network 18 and the wide area network 24 can be reassigned from lower priority duties to the emergency care center to assist with the increased volume. Additionally, by having other health care professionals, such as those that are providing “on-call” services, periodically review the emergency care center via a computer 36 connected via the wide area network 24, these additional health care professionals will be able to quickly determine whether or not their services will be required in the emergency care center. It can be appreciated by one skilled in the art that such a wide area network connection 24 provides for numerous methods of communication such as automated messages sent via e-mail, paging, text messaging, cellular telephone and land based telephone. In this manner a highly flexible and reliable system is established to enable greater workflow and resource management.

Turning now to FIG. 2, the method of the present invention is illustrated in the form a of a flow chart. The method of the present invention is directed towards an automated process for the intake and prioritization of patients as they arrive at an emergency treatment center. Clearly, the process is directed towards patients that are not arriving at the emergency treatment center with a severe or critical condition that required immediate treatment. Instead, the method of the present invention is anticipated as being directed towards approximately 50% of patients that arrive at the emergency treatments center that are conscious, not suffering from a critical condition and are capable of interacting with the interface 16 located within the kiosk 26.

In operation, the method of the present invention provides that upon entering the emergency care center 38, a patient is greeted by a health care professional 40 that is certified in triage operations. The health care professional prescreens the incoming patient and makes an initial determination 42 as to whether the arriving patient requires immediate treatment. If the arriving patient requires urgent care, the patient is immediately directed to a doctor for treatment 44. However, if the patient does not warrant immediate treatment, the health care professional directs the patient to the automated triage system 46 of the present invention, wherein the patient begins the automated triage process.

The interface 16 and monitor located in the triage kiosk 26 serve to guide the patient through a series of questions in order to complete the automated triage process 48. The patient is asked to answer questions directed at determining their background, medical history and chief complaint. As part of the process, the interface may request the preferred language, age, background and other demographic information about the patient in order to tailor the triage screening in the most appropriate manner. Further, the kiosk inquires as to the patient's medical record number or other medical identification number in order to locate any relevant prior medical history regarding this patient 49. Once the prior history is retrieved, the identity of the patient is confirmed through a series of additional questions to ensure that the record that was retrieved corresponds to the current patient.

It should be appreciated that the system monitors the condition of the patient 50 and should at any time in the automated screening process a piece of information dictate the need for immediate intervention 52 the monitor would direct the patient to seek immediate help 44 or would alert a monitoring health care professional regarding the immediacy of the need.

The system then prompts the patient to input their chief complaint. Once the chief complaint is identified the system proceed through a predetermined diagnosis tree asking more specific questions regarding the patient's condition based on the answers provided to the previous questions. In this manner, the system further narrows the tentative diagnosis of the patient thereby assisting in determining the severity of the patient's condition. For any given chief complaint given by a patient, there are a series of observations and questions that, based upon the answers given to the questions by the patient or a person who comes to the emergency department with the patient, will lead down a specific decision tree or clinical pathway. The system, by nature of the questions asked or information required, takes into consideration the patient's chief complaint, and their sex, age, vital signs, and pertinent past and present medical history in order to make a triage determination.

Accordingly, the system of the present invention provides an automated clinical pathway for making observations and obtaining relevant information in order to quickly and accurately arrive at a final triage determination. The triage level is not established by diagnosis since there is no certain diagnosis at the time of triage but is based only upon the potential for certain clinical conditions based on the presenting clinical status of the patient and the patient's symptom complex.

In conjunction with obtaining input from the patient utilizing the interface 16 and monitor 14, the system also prompts the patient to place their arm into the blood pressure cuff 30 and/or sensor array 32 so that the system can determine the current status of the patient's physiological data such as pulse, blood pressure, temperature and respiratory rate. This information is all analyzed by software that is resident in the computer processor device 12, wherein based on the totality of the information obtained, an emergency severity index (ESI) is assigned to the patient 54. All of the collected data including the ESI score is subsequently packaged into a patient record that is sent to a medical professional. Upon receipt of the medical record, the medical professional reviews the data and ranks the patients based on their ESI 56. In providing for a review of the medical record by a health care professional, it allows the introduction of professional discretion into the process of ranking patients thereby providing the ability to identify certain conditions or risk factors that may otherwise go unidentified during the automated process. Even though this system is standardized and computerized, it is not infallible. Therefore, should, upon review of the record, a health care professional believe that a patient's condition is more critical than is reflected in the ESI assigned by the system, the system can always be overridden to require a higher triage level for a patient 58, i.e. a triage level that would indicate that the patient is more critical than the triage level that was assigned to the patient by the system. In this manner, a large portion of the initial triage process is automated while still allowing the medical professionals to apply their observational skills and experience in order to make the final determination regarding the patient's condition and thereby appropriately rank patients based on their ESI 60.

Once ESI scores are assigned, the patients are referred to a waiting area where they are directed to await their turn to be seen for treatment 62. It is further possible that the system may include a monitoring device that would be worn by each patient while in the waiting area. The purpose of the monitoring device would be to provide immediate feedback to the system should a patient's physiological condition deteriorate thereby indicating a need for immediate intervention by a health care professional.

It should be appreciated that this system does not propose to dictate clinical standards for the specific time periods a patient needs to be evaluated and treated for any given ESI score. Decisions regarding the time to be seen are entirely the decision of the local medical community. Also, this system does not propose in which area of an emergency department that patients should be seen or by whom. Where a patient with a particular ESI score needs to be taken care of and who should supply that care is a decision that is best left to the individual hospital.

It can therefore be seen that the present invention provides a system and method for automating a portion of the patient intake and triage process at an emergency care center thereby providing better ability to manage the flow of patients and the need for particular staffing levels. Further, the present invention provides a method and system that serves to integrate the administrative and triage functions associated with the intake of a patient at an emergency care center while reducing the amount of staffing required to operate the triage function of the emergency care center thereby freeing valuable health care professional resources to attend to other functions. For these reasons, the present invention is believed to represent a significant advancement in the art, which has substantial commercial merit.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed:

1. An intake and prioritization system for receiving patients in an emergency care center comprising:
a system monitoring station;
at least one user interface including,
a computer processor,
software resident on said computer processor, said software configured to control the intake and prioritization process,
a means for a user to interact with said interface connected to said computer processor, wherein said user provides information regarding said patient's medical history and current medical condition in response to prompts from said software,
instruments for measuring the physiological data of said patient connected to said processor, said instruments controlled by said software, and
an electronic communication network connecting said system monitoring station and said at least one user interface,
wherein said software records and compiles said medical history, current medical condition and physiological data into a patient record,
wherein said computer processor transmits said medical record via said communication network to said monitoring station for review and prioritization.

2. The intake and prioritization system of claim 1, wherein said at least one user interface is a plurality of user interfaces, each of said user interfaces connected to said monitoring station via said communication network, wherein each of said patient records generated and transmitted by each of said user interfaces is ranked and prioritized relative to one another.

3. The intake and prioritization system of claim 1, wherein said software assigns a relative index value to each of said patent records based on the severity of the patient's current medical condition.

4. The intake and prioritization system of claim 1, said instruments for measuring the physiological data of said patient are selected from the group consisting of: a sphygmomanometer, an infrared sensor array, a means for detecting pulse rate, a means for detecting blood oxygen levels, a means for detecting body temperature, a means to measure respiratory rate and combinations thereof.

5. The intake and prioritization system of claim 1, said means for user interaction selected from the group consisting of: keyboard, mouse, touch screen monitor, monitor and combinations thereof.

6. The intake and prioritization system of claim 1, wherein said system monitoring station is a remotely located computer connected to said electronic communication network.

7. The intake and prioritization system of claim 1, further comprising:
a network accessible electronic storage device connected to said electronic communication network, said storage device containing medical records and prior medical history of a plurality of patients.

8. The intake and prioritization system of claim 1, further comprising:
a wide area electronic communication network connected to said communication network, said wide area network providing selective access to said intake and prioritization system via a plurality of remotely connected computer terminals.

9. The intake and prioritization system of claim 8, further comprising:
a plurality of network accessible electronic storage devices connected to said wide area communication network, said plurality of storage devices containing medical records and prior medical history of a plurality of patients.

10. The intake and prioritization system of claim 1, wherein said user interface is housed within a self contained kiosk, said kiosk including screening means for maintaining the privacy of the information provided by said user.

11. A method for the automation of intake and prioritization of patients at an emergency care center comprising the steps of:
receiving said patient at said emergency care center;
assessing the condition of said patient to determine the severity of the patient's medical condition;
referring high severity patients for receipt of immediate medical care;
referring low severity patients to an automated intake and prioritization system;
obtaining information from said low severity patient regarding medical history current medical condition and current physiological condition;
packaging said information into a medical record wherein said medical record is assigned a prioritization ranking;
forwarding said medical record to a monitoring station wherein said medical record is prioritized relative to all other medical records based on said prioritization ranking; and
treating said patients in order of said prioritization of said medical records.

12. The method for the automation of intake and prioritization of patients of claim 11, wherein a health care professional has discretion to refer any patient for immediate treatment based on a review of said medical record without regard for said prioritization ranking.

13. The method for the automation of intake and prioritization of patients of claim 11, wherein said step of obtaining information from said low severity patient regarding medical history current medical condition and current physiological condition includes obtaining patient medical history data from a storage device that is in electronic communication with said automated intake and prioritization system.

14. The method for the automation of intake and prioritization of patients of claim 11, said automated intake and prioritization system comprising:
a system monitoring station;
at least one user interface including,
a computer processor,
software resident on said computer processor, said software configured to control the intake and prioritization process,
a means for a user to interact with said interface connected to said computer processor, wherein said user provides information regarding said patient's medical history and current medical condition in response to prompts from said software,
instruments for measuring the physiological data of said patient connected to said processor, said instruments controlled by said software, and
an electronic communication network connecting said system monitoring station and said at least one user interface,
wherein said software records and compiles said medical history, current medical condition and physiological data into a patient record,
wherein said computer processor transmits said medical record via said communication network to said monitoring station for review and prioritization.

15. The method for the automation of intake and prioritization of patients of claim 14, wherein said system monitoring station is a remotely located computer connected to said electronic communication network.

16. The method for the automation of intake and prioritization of patients of claim 14, said system further comprising:
a network accessible electronic storage device connected to said electronic communication network, said storage device containing medical records and prior medical history of a plurality of patients.

17. The method for the automation of intake and prioritization of patients of claim 14, wherein said user interface is housed within a self contained kiosk, said kiosk including screening means for maintaining the privacy of the information provided by said user.

18. The method for the automation of intake and prioritization of patients of claim 14, said system further comprising:
a wide area electronic communication network connected to said communication network, said wide area network providing selective access to said intake and prioritization system via a plurality of remotely connected computer terminals.

19. The method for the automation of intake and prioritization of patients of claim 18, including the steps of:
monitoring the volume of patients arriving at said emergency care center via said wide area network; and
adjusting the level of health care professionals assigned to said emergency care center based on said monitoring.
